(19) United States
(12) Patent Application Publication
(10) Pub. No.: US 2021/0302113 A1
(43) Pub. Date: Sep. 30, 2021

(54) FLUORINATED DIAMINOOLEFINS AND METHODS OF USING THE SAME

(71) Applicant: 3M INNOVATIVE PROPERTIES COMPANY, St. Paul, MN (US)

(72) Inventors: Sean M. Smith, Woodbury, MN (US); Michael J. Bulinski, Stillwater, MN (US)

(21) Appl. No.: 16/496,010

(22) PCT Filed: May 15, 2018
(86) PCT No.: PCT/US2018/032651

§ 371 Date: Sep. 20, 2019

Related U.S. Application Data
(60) Provisional application No. 62/512,853, filed on May 31, 2017.

(51) Int. Cl. F28F 23/02; C07D 265/30; C07D 207/10

(52) U.S. Cl. CPC F28F 23/02; C07D 207/10; C07D 265/30

(57) ABSTRACT

Described herein is a fluorinated diaminoolefin of formula (I) (R_{f}¹CF₂)(R_{f}²)NCH₂CH═CHCH₂N(R_{f}⁴)(CF₂R_{f}³) where: R_{f}¹ and R_{f}³, are independently selected from F, a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; and R_{f}² and R_{f}⁴ are independently selected from a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof or at least one of (i) R_{f}¹CF₂ and R_{f}² and (ii) R_{f}³CF₂ and R_{f}⁴ are bonded together to form a fluorinated ring structure comprising 4-8 carbon atoms and optionally comprising at least one catenated atom selected from O, N, S or combinations thereof.

### TECHNICAL FIELD

The present disclosure relates to fluorinated diaminoolefin compounds and methods of using the same.

### SUMMARY

There continues to be a need for inert fluorinated fluids which have low global warming potential while providing high thermal stability, low toxicity, nonflammability, good solvency, and a wide operating temperature range to meet the requirements of various applications. Those applications include, but are not restricted to, heat transfer, solvent cleaning, vapor phase soldering, fire extinguishing agents, and electrolyte solvents and additives.

In one aspect, a fluorinated diaminoolefin compound of formula (I) is disclosed

(R_{f}¹CF₂)(R_{f}²)NCH₂CH═CHCH₂N(R_{f}⁴)(CF₂R_{f}³)

where:
R_{f}¹ and R_{f}³, are independently selected from F, a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; and R_{f}² and R_{f}⁴ are independently selected from a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; or
at least one of (i) R_{f}¹CF₂ and R_{f}² and (ii) R_{f}³CF₂ and R_{f}⁴ are bonded together to form a fluorinated ring structure comprising 4-8 carbon atoms and optionally comprising at least one catenated atom selected from O, N, S or combinations thereof.

In another aspect, methods of using the fluorinated diaminoolefin compound of formula (I) are disclosed, including its use in a cleaning composition, as an electrolyte solvent, a heat transfer fluid, or a vapor phase soldering fluid.

In another aspect, the fluorinated diaminoolefin compound of formula (I) is used in a heat transfer apparatus.

The above summary is not intended to describe each embodiment. The details of one or more embodiments of the invention are also set forth in the description below. Other features, objects, and advantages will be apparent from the description and from the claims.

### DETAILED DESCRIPTION

As used herein, the term

“a”, “an”, and “the” are used interchangeably and mean one or more; and

“and/or” is used to indicate one or both stated cases may occur, for example A and/or B includes, (A and B) and (A or B);

“alkyl” refers to a monovalent group that is a radical of an alkane, which is a saturated hydrocarbon. The alkyl group can be linear, branched, cyclic or combinations thereof;

“catenated” means an atom other than carbon (for example, oxygen or nitrogen) that is bonded to at least two carbon atoms in a carbon chain (linear or branched or within a ring) so as to form a carbon-heteroatom-carbon linkage; and

“perfluorinated” means a group or a compound wherein all hydrogen atoms in the C—H bonds have been replaced by C—F bonds.

As used herein, a chemical structure that depicts the letter “F” in the center of a ring indicates that all unmarked bonds of the ring are fluorine atoms.

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

Heat transfer fluids may be used to transmit heat from one location to another, for example, to prevent over heating of a device or to maintain precise temperature control or for energy conversion, as in the capture of waste heat and the conversion to electrical or mechanical energy. Presently, various fluids are used for heat transfer. The suitability of the heat transfer fluid depends upon the application process. For example, in some electronic applications, a heat-transfer fluid which is inert, has low toxicity, good environmental properties, and good heat transfer properties over a wide temperature range is desirable.

Vapor phase soldering is a process application that requires heat transfer fluids which are especially suitable for high temperature exposure. In such application, temperatures of between 170° C. and 250° C. are typically used with 200° C. being particularly useful for soldering applications using a lead based solder and 230° C. useful for the higher melting lead free solders. Currently, the heat transfer fluids used in vapor phase soldering are of the perfluoropolyether (PFPE) class. While many PFPEs have adequate thermal stability at the temperatures employed, they also possess the notable drawback of being environmentally persistent with extremely long atmospheric lifetimes which, in turn, gives rise to high global warming potentials (GWPs). As such, there is a need for new materials which possess the characteristics of the PFPEs that make them useful in vapor phase soldering as well as in other high temperature heat transfer applications (e.g., chemical inertness, thermal stability and effective heat transfer, liquid over a wide temperature range, good heat-transfer properties over a wide range of temperatures), but which have a much shorter atmospheric lifetime and lower GWPs.

In the present disclosure, it has been discovered that the compounds of the present disclosure can have short atmospheric lifetimes (compared to perfluorinated hydrocarbons and hydrofluoro carbons) while being stable at elevated temperatures, such as the temperatures experienced in vapor phase soldering.

In some embodiments, the fluorinated diaminoolefin compound of the present disclosure may exhibit properties that render them particularly useful as heat transfer fluids for the electronics industry. For example, the fluorinated diaminoolefin compound may be chemically inert (i.e., they do not easily react with base, acid, water, etc.), and may have high boiling points (up to 300° C.), low freezing points (the fluorinated diaminoolefin compound may be liquid at −40° C. or lower), low viscosity, high thermal stability, good thermal conductivity, adequate solvency for a range of potentially important solutes, and low toxicity. The fluorinated diaminoolefin compound of the present disclosure may also, surprisingly, be liquid at room temperature (e.g., between 20 and 25° C.).

Further, in one embodiment, the compounds of the present disclosure can be readily prepared in high yield via low cost starting materials. The starting materials can be readily purchased or derived from electrochemical fluorination. Thus, the compounds described in the present disclosure represent a new class of useful and potentially low cost fluorinated fluids that offer potential advantages in a variety of applications including heat transfer, cleaning, and electrolyte applications.

The fluorinated diaminoolefin compound of the present disclosure (herein referred to interchangeably as a compound of the present disclosure) are of the general formula (I)

(R_{f}¹CF₂)(R_{f}²)NCH₂CH═CHCH₂N(R_{f}⁴)(CF₂R_{f}³)

where:
R_{f}¹ and R_{f}³, are independently selected from F, a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; and R_{f}² and R_{f}⁴ are independently selected from a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; and/or at least one of (i) R_{f}¹CF₂ and R_{f}² and (ii) R_{f}³CF₂ and R_{f}⁴ are bonded together to form a fluorinated ring structure comprising a total of 4-8 carbon atoms and optionally comprising at least one catenated atom selected from O, N, S or combinations thereof.

It is to be appreciated that the fluorinated diaminoolefin compounds of the present disclosure may include the cis isomer, the trans isomer, or a mixture of cis and trans isomers.

In one embodiment, the R_{f}¹, R_{f}², R_{f}³, and R_{f}⁴ are independently selected from a linear or branched perfluorinated alkyl group comprising 1-4 carbon atoms and optionally comprising at least one catenated atom selected from O, N, S or combinations thereof. In one embodiment, the R_{f}¹, R_{f}², R_{f}³, and R_{f}⁴ are independently selected from a linear or branched perfluorinated alkyl group comprising 1 or 2 carbon atoms.

Exemplary R_{f}¹, R_{f}², R_{f}³, and R_{f}⁴ include: —CF₃, —CF₂CF₃, —CF₂CF₂CF₃, —CF(CF₃)₂, and —CF₂OCF₂CF₃.

In one embodiment, the R_{f}¹CF₂ and R_{f}² are bonded together to form a fluorinated ring structure comprising 4 to 8 carbon atoms. The ring is a 5, 6, 7, or 8-membered ring, which includes the nitrogen atom. The fluorinated ring structure comprises a total of 4 to 8 carbon atoms, wherein the carbon atoms may be a ring member or in substituents off of the ring (such as alkyl groups). The fluorinated ring structure may optionally comprise at least one catenated atom selected from O, N, S or combinations thereof. The catenated atoms may be a ring member or located in a substituent off of the ring.

In one embodiment, the R_{f}³CF₂ and R_{f}⁴ are bonded together to form a fluorinated ring structure comprising 4 to 8 carbon atoms. The ring is a 5, 6, 7, or 8-membered ring, which includes the nitrogen atom. The fluorinated ring structure comprises a total of 4 to 8 carbon atoms, wherein the carbon atoms may be a ring member or in substituents off of the ring (such as alkyl groups). The fluorinated ring structure may optionally comprise at least one catenated atom selected from O, N, S or combinations thereof. The catenated atoms may be a ring member or located in a substituent off of the ring.

In one embodiment, the cyclic N(CF₂R_{f}¹)(R_{f}²) group and/or the cyclic N(CF₂R_{f}³)(R_{f}⁴) group are bonded together to form a 6-membered ring comprising an additional catenated nitrogen atom, forming for example a perfluorinated piperazine ring, wherein the additional nitrogen heteroatom is tertiary and is bonded to a perfluoroalkyl group having 1-3 carbon atoms. In one embodiment, the cyclic N(CF₂R_{f}¹)(R_{f}²) group and/or the cyclic N(CF₂R_{f}³)(R_{f}⁴) group are bonded together to form a perfluorinated pyrrolidine group. In one embodiment, the cyclic N(CF₂R_{f}¹)(R_{f}²) group and/or the cyclic N(CF₂R_{f}³)(R_{f}⁴) group are bonded together to form a 6-membered ring comprising a catenated O atom, forming for example a perfluorinated morpholine ring.

Exemplary cyclic N(CF₂R_{f}¹)(R_{f}²) group and/or the cyclic N(CF₂R_{f}³)(R_{f}⁴) groups include:

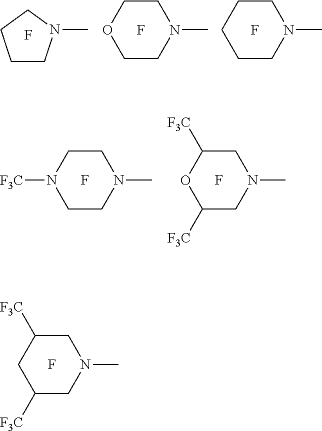

In one embodiment, the fluorinated diaminoolefin compound is symmetric around the double bond, meaning that the N(CF₂R_{f}¹)(R_{f}²) group and the N(CF₂R_{f}³)(R_{f}⁴) group are the same. In one embodiment, the N(CF₂R_{f}¹)(R_{f}²) group and the N(CF₂R_{f}³)(R_{f}⁴) groups are different.

Exemplary fluorinated diaminoolefin compound of the present disclosure include the following compounds:

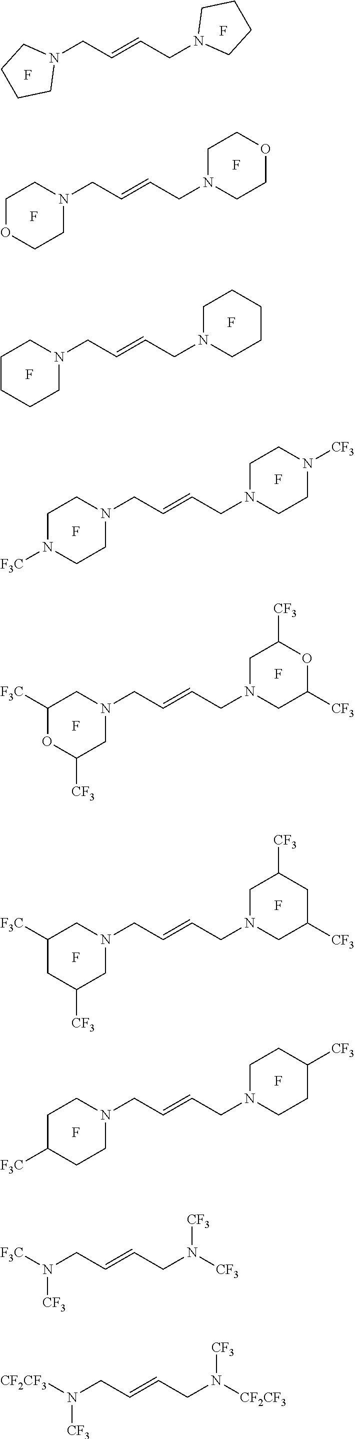

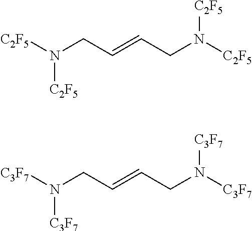

Although the above compounds are drawn in a trans configuration, it is noted that that the fluorinated diaminoolefin compounds can be in the cis configuration, or mixtures of cis and trans compounds.

The compounds of the present disclosure have good environmental properties as well as having good performance attributes, such as non-flammability, chemical inertness, high thermal stability, good solvency, etc.

In one embodiment, the compound of the present disclosure may have a low environmental impact. In this regard, the compounds of the present disclosure may have a global warming potential (GWP) of less than 100, 50, or even 10. As used herein, GWP is a relative measure of the global warming potential of a compound based on the structure of the compound. The GWP of a compound, as defined by the Intergovernmental Panel on Climate Change (IPCC) in 1990 and updated in 2007, is calculated as the warming due to the release of 1 kilogram of a compound relative to the warming due to the release of 1 kilogram of CO₂ over a specified integration time horizon (ITH).

$${GWP}_{i} ( {t}^{′} ) = \frac{{\int }_{0}^{I T H} {a}_{i} [ C ( t ) ] d t}{{{\int }^{ITH}}_{0} {a}_{CO 2} [ {C}_{CO 2} ( t ) ] dt} = \frac{{\int }_{0}^{I T H} {a}_{i} {C}_{oi} {e}^{- t / {τ}_{ι}} d t}{{{\int }^{ITH}}_{0} {a}_{CO 2} [ {C}_{CO 2} ( t ) ] dt}$$

In this equation a, is the radiative forcing per unit mass increase of a compound in the atmosphere (the change in the flux of radiation through the atmosphere due to the IR absorbance of that compound), C is the atmospheric concentration of a compound, τ is the atmospheric lifetime of a compound, t is time, and i is the compound of interest. The commonly accepted ITH is 100 years representing a compromise between short-term effects (20 years) and longer-term effects (500 years or longer). The concentration of an organic compound, i, in the atmosphere is assumed to follow pseudo first order kinetics (i.e., exponential decay). The concentration of CO₂ over that same time interval incorporates a more complex model for the exchange and removal of CO₂ from the atmosphere (the Bern carbon cycle model).

In one embodiment, the compounds of the present disclosure have atmospheric lifetime of less than 1 year, 0.5 years, or even less than 0.1 years.

Non-flammability can be assessed by using standard methods such as ASTM D-3278-96 e-1, D56-05 “Standard Test Method for Flash Point of Liquids by Small Scale Closed-Cup Apparatus”. In one embodiment, the compound of the present disclosure is non-flammable based on closed-cup flashpoint testing following ASTM D-327-96 e-1.

In one embodiment, the compound of the present disclosure is non-bioaccumulative in animal tissues. For example, some compounds of the present disclosure may provide low log K_{ow} values, indicating a reduced tendency to bioaccumulate in animal tissues, where K_{ow} is the octanol/water partition coefficient, which is defined as the ratio of the given compound's concentration in a two-phase system comprising an octanol phase and an aqueous phase. In one embodiment, the log K_{ow} value is less than 7, 6, 5, or even 4.

In one embodiment, the compound of the present disclosure is expected to provide low acute toxicity based on 4 hour acute inhalation or oral toxicity studies in rats following U.S. EPA “Health Effects Test Guidelines OPPTS 870.1100 Acute Oral Toxicity” and/or OECD Test No. 436 “Acute Inhalation Toxicity—Acute Toxic Class Method”. For example, a compound of the present disclosure has a single dose oral median lethal dose (LD 50) in male and female Sprague-Dawley rats of greater than 30, 50, 100, 200, or even 300 mg/kg.

The useful liquid range of a compound of the present disclosure is between its pour point and its boiling point. A pour point is the lowest temperature at which the compound is still able to be poured. The pour point can be determined, for example, by ASTM D 97-16 “Standard Test Method for Pour Point of Petroleum Products”. In one embodiment, the compound of the present disclosure has a pour point of less than 15° C., 0° C., −20° C., −40° C. or even −60° C. In one embodiment, the compound of the present disclosure has a boiling point of at least 100° C., 150° C., or even at least 200° C.; and at most 250° C. or even at most 300° C.

In some embodiments, the compound of the present disclosure may be hydrophobic, relatively chemically unreactive, and thermally stable.

In one embodiment, the compound of the present disclosure reducing the potential for dehydrofluorination of the compound at elevated temperatures due to the presence of the nitrogen. In one embodiment, the compound of the present disclosure is thermally stable, meaning that when the compound is heated near its boiling point (i.e., within 5° C.) for at least 12 hours and no more than 36 hours and is subsequently analyzed by GC-FID the difference in the purity of the compound before and after heating does not change by more than 5, 2, 1, 0.5, or even 0.1%.

In one embodiment, the compound of the present disclosure may be prepared by substitution of a halogenated butane by a perfluorinated imine in the presence of a fluoride ion (F⁻).

The halogenated butane is a 1,4-dihalogenated 2-butene such as 1,4-dibromo-2-butene, 1-chloro-4-bromo-2-butene, 1,4-dichloro-2-butene, 1,4-diiodo-2-butene, or mixtures thereof.

The perfluorinated imine is of the formula (R_{f})FC═N(R_{f}¹) where

(i) R_{f} is selected from F; a linear or branched perfluorinated alkyl group comprising 1-7 carbon atoms; or a linear or branched perfluorinated alkyl group comprising at least one catenated atom selected from O, N, S or combinations thereof; and
R_{f}¹ is selected from a linear or branched perfluorinated alkyl group comprising 1-7 carbon atoms; or a linear or branched perfluorinated alkyl group comprising at least one catenated atom selected from O, N, S or combinations thereof; or
   (ii) R_{f} and R_{f}¹ are bonded together to form (a) a fluorinated ring structure comprising a total of 4-8 carbon atoms, or (b) a fluorinated ring structure comprising a total of 4-8 carbon atoms and comprising at least one catenated atom selected from O, N, S or combinations thereof.

When R_{f} and R_{f}¹ are bonded together to form a fluorinated ring structure, the ring is a 5, 6, 7, or 8-membered ring, which includes the imine nitrogen. The fluorinated ring structure comprises a total of 4 to 8 carbon atoms, wherein the carbons may be part of the ring member or substituents off of the ring (such as alkyl groups). The fluorinated ring structure may optionally comprise at least one catenated atom selected from O, N, S or combinations thereof. The catenated atoms, may be a ring member or located in a substituent off of the ring.

Exemplary perfluorinated imines include:

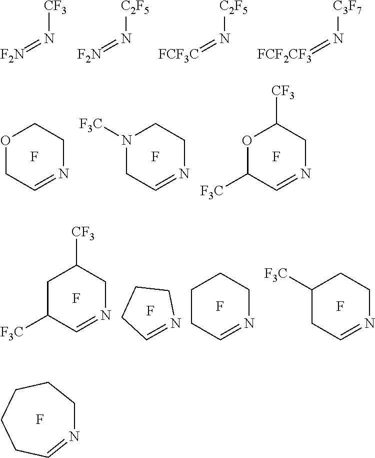

Such perfluorinated imines may be synthesized as described in H. V. Rasika Dias et al. Dalton Trans. 2011, 40, 8569; V. A. Petrov, G. G. Belen'kii, L. S. German Bulletin of the Academy of Sciences of the USSR Division of Chemical Science 1985 34, 1789; and V. A. Petrov et al. Izvestiya Akademii Nauk SSSR, Seriya Khimicheskaya 1989, 1, 122.

The mole ratio of the perfluorinated imines to the halogenated butene may be from 6:1 to 2:1, more preferably 3:1 to 2:3. Generally, at least two moles of perfluorinated imines are required per one mole of halogenated butene to achieve the compound of the present disclosure. In one embodiment, a single perfluorinated imine is used in the reaction. Alternatively, two or more different perfluorinated imines may be used in the reaction, resulting in a mixture of fluorinated diaminoolefins, which may be advantageous when targeting a molecule with a particular boiling point.

The addition of a fluoride ion to the perfluorinated imine can form an intermediate tertiary fluorocarbanion. This fluorocarbonanion can then undergo an alkylation reaction with the halogenated butene to form the compound of the present disclosure as shown in the exemplary equation below.

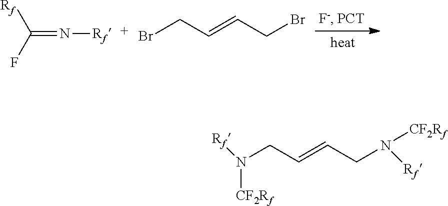

Wherein R_{f} and R_{f}¹ are defined above, and PCT refers to a phase transfer catalyst. The above equation is for illustrative purposes only, for example, cis or trans isomers may be formed.

Sources of a fluoride ion include, for example, metal salts of fluoride such as alkali and alkaline earth metal fluoride salts. Exemplary metal salts include KF, KI, CsF, AgF, and CuF. The amount of fluoride ion may be at least a stoichiometric amount, i.e., one mole of perfluorinated imine requires one mole or more of fluoride ion.

A solvent and phase transfer catalyst may be used to sufficiently dissolve the fluorocarboanion and the halogenated butane for a reaction to occur. Phase transfer catalysts are generally known in the art and include, for example, quaternary ammonium salts, such as tricaprylylmethyl ammonium chloride (also known as aliquat 336), trioctylmethylammonium chloride, benzyltrimethylammonium bromide, benzyltriethylammonium chloride and tetrabutylammonium hydrogen sulfate; quaternary phosphonium salts such as tetraphenylphosphonium chloride and trioctylethylphosphonium bromide; and crown ethers such as 18-crown-6, diaza-18-crown-6, and dibenzo-18-crown-6. Solvents include those that exhibit both lipophilic and hydrophilic properties, such as polar aprotic solvents. Polar aprotic solvents include alcohols (such as methanol and ethanol), ketones (such as acetone and dioxane), ethers (such as bis(2-methoxyethyl) ether and tetraethylene glycol dimethyl ether), nitriles (such as acetonitrile and benzonitrile), dimethylsulfoxide, N-methylpyrrolidinone (NMP), dichloromethane, N,N-dimethylformamide (DMF), tetrahydrothiophene-1,1-dioxide (sulfolane), and dimethyl sulfone, which can be used individually or as a mixture.

The reaction of the halogenated butene with the perfluorinated imine can be conducted at ambient pressures in the presence of heat. In one embodiment, the reaction of the halogenated butene with the perfluorinated imine is conducted in a sealed vessel in the presence of heat, resulting in increased pressures. In some embodiments, the halogenated butene and the perfluorinated imine are heated along with the metal fluoride and phase transfer catalyst at temperatures higher than ambient (such as at least 50° C.) and no greater than 75, 100, 125, or even 150° C.

In one embodiment, the resulting fluorinated compounds can be purified to isolate the desired diaminoolefin. Purification can be done by conventional means including distillation, absorption, extraction, chromatography and recrystallization. The purification can be done to isolate the compound of the present disclosure (in all its distereoisomeric forms) from impurities, such as starting materials, byproducts, etc. The term “purified form” as used herein means the compound of the present disclosure is at least 90, 95, 98, or even 99 wt % pure.

The compounds of the present disclosure may be used as a working fluid in a variety of applications. The working fluids may include at least 25%, 50%, 70%, 80%, 90%, 95%, 99%, or even 100% by weight of the above-described formula (I) compounds based on the total weight of the working fluid. In addition to the compounds of the present disclosure, the working fluids may include a total of up to 75%, up to 50%, up to 30%, up to 20%, up to 10%, or up to 5% by weight of one or more of the following components: alcohols, ethers, alkanes, alkenes, haloalkenes, perfluorocarbons, perfluorinated tertiary amines, perfluoroethers, cycloalkanes, esters, ketones, oxiranes, aromatics, siloxanes, unsaturated hydrochlorocarbons, unsaturated hydrochlorofluorocarbons, unsaturated hydrofluorocarbons, non-hetero atom-containing hydrofluoroolefins, hydrochloroolefins, hydrochlorofluoroolefins, unsaturated hydrofluoroethers, or mixtures thereof, based on the total weight of the working fluid. Such additional components can be chosen to modify or enhance the properties of a composition for a particular use.

In one embodiment, the working fluid has no flash point (as measured, for example, following ASTM D-3278-96 e-1).

In one embodiment, the compound of the present disclosure may be used in an apparatus for heat transfer that includes a device and a mechanism for transferring heat to or from the device. The mechanism for transferring heat may include a heat transfer working fluid that includes a compound of formula (I) of the present disclosure.

The provided apparatus for heat transfer may include a device. The device may be a component, work-piece, assembly, etc. to be cooled, heated or maintained at a predetermined temperature or temperature range. Such devices include electrical components, mechanical components and optical components. Examples of devices of the present disclosure include, but are not limited to microprocessors, wafers used to manufacture semiconductor devices, power control semiconductors, electrical distribution switch gear, power transformers, circuit boards, multi-chip modules, packaged and unpackaged semiconductor devices, lasers, chemical reactors, fuel cells, and electrochemical cells. In some embodiments, the device can include a chiller, a heater, or a combination thereof.

In yet other embodiments, the devices can include electronic devices, such as processors, including microprocessors. As these electronic devices become more powerful, the amount of heat generated per unit time increases. Therefore, the mechanism of heat transfer plays an important role in processor performance. The heat-transfer fluid typically has good heat transfer performance, good electrical compatibility (even if used in “indirect contact” applications such as those employing cold plates), as well as low toxicity, low (or non-) flammability and low environmental impact. Good electrical compatibility suggests that the heat-transfer fluid candidate exhibit high dielectric strength, high volume resistivity, and poor solvency for polar materials. Additionally, the heat-transfer fluid should exhibit good mechanical compatibility, that is, it should not affect typical materials of construction in an adverse manner.

The provided apparatus may include a mechanism for transferring heat. The mechanism may include a heat transfer fluid. The heat transfer fluid may include one or more compounds of the present disclosure. Heat may be transferred by placing the heat transfer mechanism in thermal contact with the device. The heat transfer mechanism, when placed in thermal contact with the device, removes heat from the device or provides heat to the device, or maintains the device at a selected temperature or temperature range. The direction of heat flow (from device or to device) is determined by the relative temperature difference between the device and the heat transfer mechanism.

The heat transfer mechanism may include facilities for managing the heat-transfer fluid, including, but not limited to pumps, valves, fluid containment systems, pressure control systems, condensers, heat exchangers, heat sources, heat sinks, refrigeration systems, active temperature control systems, and passive temperature control systems. Examples of suitable heat transfer mechanisms include, but are not limited to, temperature controlled wafer chucks in plasma enhanced chemical vapor deposition (PECVD) tools, temperature-controlled test heads for die performance testing, temperature-controlled work zones within semiconductor process equipment, thermal shock test bath liquid reservoirs, and constant temperature baths. In some systems, such as etchers, ashers, PECVD chambers, vapor phase soldering devices, and thermal shock testers, the upper desired operating temperature may be as high as 170° C., as high as 200° C., or even as high as 230° C.

Heat can be transferred by placing the heat transfer mechanism in thermal contact with the device. The heat transfer mechanism, when placed in thermal contact with the device, removes heat from the device or provides heat to the device, or maintains the device at a selected temperature or temperature range. The direction of heat flow (from device or to device) is determined by the relative temperature difference between the device and the heat transfer mechanism. The provided apparatus can also include refrigeration systems, cooling systems, testing equipment and machining equipment. In some embodiments, the provided apparatus can be a constant temperature bath or a thermal shock test bath. In some systems, such as etchers, ashers, PECVD chambers, vapor phase soldering devices, and thermal shock testers, the upper desired operating temperature may be as high as 170° C., as high as 200° C., 250° C. or even higher.

In some embodiments, the compounds of the present disclosure may be used as a heat transfer agent for use in vapor phase soldering. In using the compounds of the present disclosure vapor phase soldering, the process described in, for example, U.S. Pat. No. 5,104,034 (Hansen) can be used, which description is hereby incorporated by reference. Briefly, such process includes immersing a component to be soldered in a body of vapor comprising at least a fluorinated diaminoolefin compound of the present disclosure to melt the solder. In carrying out such a process, a liquid pool of the fluorinated diaminoolefin compound (or working fluid that includes the fluorinated diaminoolefin compound) is heated to boiling in a tank to form a saturated vapor in the space between the boiling liquid and a condensing means.

A workpiece to be soldered is immersed in the vapor (at a temperature of greater than 170° C., greater than 200° C., greater than 230° C., 250° C., or even greater), whereby the vapor is condensed on the surface of the workpiece so as to melt and reflow the solder. Finally, the soldered workpiece is then removed from the space containing the vapor.

In another embodiment, the compound of the present disclosure is used in an apparatus for converting thermal energy into mechanical energy in a Rankine cycle. The apparatus may include a working fluid that includes one or more compounds of formula (I). The apparatus may further include a heat source to vaporize the working fluid and form a vaporized working fluid, a turbine through which the vaporized working fluid is passed thereby converting thermal energy into mechanical energy, a condenser to cool the vaporized working fluid after it is passed through the turbine, and a pump to recirculate the working fluid.

In some embodiments, the present disclosure relates to a process for converting thermal energy into mechanical energy in a Rankine cycle. The process may include using a heat source to vaporize a working fluid that includes one or more compounds of formula (I) to form a vaporized working fluid. In some embodiments, the heat is transferred from the heat source to the working fluid in an evaporator or boiler. The vaporized working fluid may be pressurized and can be used to do work by expansion. The heat source can be of any form such as from fossil fuels, e.g., oil, coal, or natural gas. Additionally, in some embodiments, the heat source can come from nuclear power, solar power, or fuel cells. In other embodiments, the heat can be “waste heat” from other heat transfer systems that would otherwise be lost to the atmosphere. The “waste heat,” in some embodiments, can be heat that is recovered from a second Rankine cycle system from the condenser or other cooling device in the second Rankine cycle.

An additional source of “waste heat” can be found at landfills where methane gas is flared off. In order to prevent methane gas from entering the environment and thus contributing to global warming, the methane gas generated by the landfills can be burned by way of “flares” producing carbon dioxide and water which are both less harmful to the environment in terms of global warming potential than methane. Other sources of “waste heat” that can be useful in the provided processes are geothermal sources and heat from other types of engines such as gas turbine engines that give off significant heat in their exhaust gases and to cooling liquids such as water and lubricants.

In the provided process, the vaporized working fluid may be expanded though a device that can convert the pressurized working fluid into mechanical energy. In some embodiments, the vaporized working fluid is expanded through a turbine which can cause a shaft to rotate from the pressure of the vaporized working fluid expanding. The turbine can then be used to do mechanical work such as, in some embodiments, operate a generator, thus generating electricity. In other embodiments, the turbine can be used to drive belts, wheels, gears, or other devices that can transfer mechanical work or energy for use in attached or linked devices.

After the vaporized working fluid has been converted to mechanical energy the vaporized (and now expanded) working fluid can be condensed using a cooling source to liquefy for reuse. The heat released by the condenser can be used for other purposes including being recycled into the same or another Rankine cycle system, thus saving energy. Finally, the condensed working fluid can be pumped by way of a pump back into the boiler or evaporator for reuse in a closed system.

The desired thermodynamic characteristics of organic Rankine cycle working fluids are well known to those of ordinary skill and are discussed, for example, in U.S. Pat. Appl. Publ. No. 2010/0139274 (Zyhowski et al.). The greater the difference between the temperature of the heat source and the temperature of the condensed liquid or a provided heat sink after condensation, the higher the Rankine cycle thermodynamic efficiency. The thermodynamic efficiency is influenced by matching the working fluid to the heat source temperature. The closer the evaporating temperature of the working fluid to the source temperature, the higher the efficiency of the system. Toluene can be used, for example, in the temperature range of 79° C. to about 260° C., however toluene has toxicological and flammability concerns. Fluids such as 1,1-dichloro-2,2,2-trifluoroethane and 1,1,1,3,3-pentafluoropropane can be used in this temperature range as an alternative. 1,1-dichloro-2,2,2-trifluoroethane can form toxic compounds below 300° C. and should to be limited to an evaporating temperature of about 93° C. to about 121° C. Thus, there is a desire for other environmentally-friendly Rankine cycle working fluids with higher critical temperatures so that source temperatures such as gas turbine and internal combustion engine exhaust can be better matched to the working fluid.

In one embodiment, the compound of the present disclosure is used in a cleaning composition along with one or more co-solvents. In some embodiments, the present disclosure relates to a process for cleaning a substrate. The cleaning process can be carried out by contacting a contaminated substrate with a cleaning composition. The compound of the present disclosure can be utilized alone or in admixture with each other or with other commonly-used cleaning co-solvents. Representative examples of co-solvents which can be used in the cleaning composition include methanol, ethanol, isopropanol, t-butyl alcohol, methyl t-butyl ether, methyl t-amyl ether, 1,2-dimethoxyethane, cyclohexane, 2,2,4-trimethylpentane, n-decane, terpenes (e.g., a-pinene, camphene, and limonene), trans-1,2-dichloroethylene, cis-1,2-dichloroethylene, methylcyclopentane, decalin, methyl decanoate, t-butyl acetate, ethyl acetate, diethyl phthalate, 2-butanone, methyl isobutyl ketone, naphthalene, toluene, p-chlorobenzotrifluoride, trifluorotoluene, bis(trifluoromethyl)benzenes, hexamethyl disiloxane, octamethyl trisiloxane, perfluorohexane, perfluoroheptane, perfluorooctane, perfluorotributylamine, perfluoro-N-methyl morpholine, perfluoro-2-butyl oxacyclopentane, methylene chloride, chlorocyclohexane, 1-chlorobutane, 1,1-dichloro-1-fluoroethane, 1,1,1-trifluoro-2,2-dichloroethane, 1,1,1,2,2-pentafluoro-3,3-dichloropropane, 1,1,2,2,3-pentafluoro-1,3-dichloropropane, 2,3-dihydroperfluoropentane, 1,1,1,2,2,4-hexafluorobutane, 1-trifluoromethyl-1,2,2-trifluorocyclobutane, 3-methyl-1,1,2,2-tetrafluorocyclobutane, 1-hydropentadecafluoroheptane, or mixtures thereof. Such co-solvents can be chosen to modify or enhance the solvency properties of a cleaning composition for a particular use and can be utilized in ratios (of co-solvent to compounds according to formula (I)) such that the resulting composition has no flash point. If desirable for a particular application, the cleaning composition can further contain one or more dissolved or dispersed gaseous, liquid, or solid additives (for example, carbon dioxide gas, surfactants, stabilizers, antioxidants, or activated carbon).

In some embodiments, the present disclosure relates to cleaning compositions that include one or more compounds of the present disclosure and optionally one or more surfactants. Suitable surfactants include those surfactants that are sufficiently soluble in the compound of the present disclosure, and which promote soil removal by dissolving, dispersing or displacing the soil. One useful class of surfactants are those nonionic surfactants that have a hydrophilic-lipophilic balance (HLB) value of less than about 14. Examples include ethoxylated alcohols, ethoxylated alkylphenols, ethoxylated fatty acids, alkylaryl sulfonates, glycerol esters, ethoxylated fluoroalcohols, and fluorinated sulfonamides. Mixtures of surfactants having complementary properties may be used in which one surfactant is added to the cleaning composition to promote oily soil removal and another added to promote water-soluble soil removal. The surfactant, if used, can be added in an amount sufficient to promote soil removal. Typically, surfactant may be added in amounts from 0.1 to 5.0 wt. % or from 0.2 to 2.0 wt. % of the cleaning composition.

The cleaning compositions can be used in either the gaseous or the liquid state (or both), and any of known or future techniques for “contacting” a substrate can be utilized. For example, a liquid cleaning composition can be sprayed or brushed onto the substrate, a gaseous cleaning composition can be blown across the substrate, or the substrate can be immersed in either a gaseous or a liquid composition. Elevated temperatures, ultrasonic energy, and/or agitation can be used to facilitate the cleaning. Various different solvent cleaning techniques are described by B. N. Ellis in Cleaning and Contamination of Electronics Components and Assemblies, Electrochemical Publications Limited, Ayr, Scotland, pages 182-94 (1986).

Both organic and inorganic substrates can be cleaned by the processes of the present disclosure. Representative examples of the substrates include metals; ceramics; glass; polycarbonate; polystyrene; acrylonitrile-butadiene-styrene copolymer; natural fibers (and fabrics derived therefrom) such as cotton, silk, fur, suede, leather, linen, and wool; synthetic fibers (and fabrics) such as polyester, rayon, acrylics, nylon, or blends thereof; fabrics comprising a blend of natural and synthetic fibers; and composites of the foregoing materials. In some embodiments, the process may be used in the precision cleaning of electronic components (e.g., circuit boards), optical or magnetic media, or medical devices.

In still another embodiment, the compound of the present disclosure is used in dielectric fluids, which can be used in electrical devices (e.g., capacitors, switchgear, transformers, or electric cables or buses). For purposes of the present application, the term “dielectric fluid” is inclusive of both liquid dielectrics and gaseous dielectrics. The physical state of the fluid, gas or liquid, is determined at the operating conditions of temperature and pressure of the electrical device in which it is used.

In some embodiments, the dielectric fluids include one or more compounds of formula (I) and, optionally, one or more second dielectric fluids. Suitable second dielectric fluids include, for example, air, nitrogen, helium, argon, and carbon dioxide, or combinations thereof. The second dielectric fluid may be a non-condensable gas or an inert gas. Generally, the second dielectric fluid may be used in amounts such that vapor pressure is at least 70 kPa at 25° C., or at the operating temperature of the electrical device.

The dielectric fluids of the present application comprising the compounds of formula (I) are useful for electrical insulation and for arc quenching and current interruption equipment used in the transmission and distribution of electrical energy. Generally, there are three major types of electrical devices in which the fluids of the present disclosure can be used: (1) gas-insulated circuit breakers and current-interruption equipment, (2) gas-insulated transmission lines, and (3) gas-insulated transformers. Such gas-insulated equipment is a major component of power transmission and distribution systems.

In some embodiments, the present disclosure provides electrical devices, such as capacitors, comprising metal electrodes spaced from each other such that the gaseous dielectric fills the space between the electrodes. The interior space of the electrical device may also comprise a reservoir of the liquid dielectric fluid which is in equilibrium with the gaseous dielectric fluid. Thus, the reservoir may replenish any losses of the dielectric fluid.

In another embodiment, the present disclosure relates to coating compositions comprising (a) a solvent composition that includes one or more compounds of the present disclosure, and (b) one or more coating materials which are soluble or dispersible in the solvent composition.

In various embodiments, the coating materials of the coating compositions may include pigments, lubricants, stabilizers, adhesives, anti-oxidants, dyes, polymers, pharmaceuticals, release agents, inorganic oxides, and the like, and combinations thereof. For example, coating materials may include unsaturated perfluoropolyether, unsaturated hydrocarbon, and silicone lubricants; amorphous copolymers of tetrafluoroethylene; polytetrafluoroethylene; or combinations thereof. Further examples of suitable coating materials include titanium dioxide, iron oxides, magnesium oxide, unsaturated perfluoropolyethers, polysiloxanes, stearic acid, acrylic adhesives, polytetrafluoroethylene, amorphous copolymers of tetrafluoroethylene, or combinations thereof.

In some embodiments, the above-described coating compositions can be useful in coating deposition, where the compounds of Formula (I) function as a carrier for a coating material to enable deposition of the material on the surface of a substrate. In this regard, the present disclosure further relates to a process for depositing a coating on a substrate surface using the coating composition. The process comprises the step of applying to at least a portion of at least one surface of a substrate a coating of a liquid coating composition comprising (a) a solvent composition containing one or more of the compounds of formula (I); and (b) one or more coating materials which are soluble or dispersible in the solvent composition. The solvent composition can further comprise one or more co-dispersants or co-solvents and/or one or more additives (e.g., surfactants, coloring agents, stabilizers, anti-oxidants, flame retardants, and the like). Preferably, the process further comprises the step of removing the solvent composition from the coating by, e.g., allowing evaporation (which can be aided by the application of, e.g., heat or vacuum).

In various embodiments, to form a coating composition, the components of the coating composition (i.e., the compound(s) of formula (I), the coating material(s), and any co-dispersant(s) or co-solvent(s) utilized) can be combined by any conventional mixing technique used for dissolving, dispersing, or emulsifying coating materials, e.g., by mechanical agitation, ultrasonic agitation, manual agitation, and the like. The solvent composition and the coating material(s) can be combined in any ratio depending upon the desired thickness of the coating. For example, the coating material(s) may constitute from about 0.1 to about 10 weight percent of the coating composition.

In illustrative embodiments, the deposition process of the disclosure can be carried out by applying the coating composition to a substrate by any conventional technique. For example, the composition can be brushed or sprayed (e.g., as an aerosol) onto the substrate, or the substrate can be spin-coated. In some embodiments, the substrate may be coated by immersion in the composition. Immersion can be carried out at any suitable temperature and can be maintained for any convenient length of time. If the substrate is a tubing, such as a catheter, and it is desired to ensure that the composition coats the lumen wall, the composition may be drawn into the lumen by the application of reduced pressure.

In various embodiments, after a coating is applied to a substrate, the solvent composition can be removed from the coating (e.g., by evaporation). If desired, the rate of evaporation can be accelerated by application of reduced pressure or mild heat. The coating can be of any convenient thickness, and, in practice, the thickness will be determined by such factors as the viscosity of the coating material, the temperature at which the coating is applied, and the rate of withdrawal (if immersion is utilized).

Both organic and inorganic substrates can be coated by the processes of the present disclosure. Representative examples of the substrates include metals, ceramics, glass, polycarbonate, polystyrene, acrylonitrile-butadiene-styrene copolymer, natural fibers (and fabrics derived therefrom) such as cotton, silk, fur, suede, leather, linen, and wool, synthetic fibers (and fabrics) such as polyester, rayon, acrylics, nylon, or blends thereof, fabrics including a blend of natural and synthetic fibers, and composites of the foregoing materials. In some embodiments, substrates that may be coated include, for example, magnetic hard disks or electrical connectors with perfluoropolyether lubricants or medical devices with silicone lubricants.

In some embodiments, the present disclosure further relates to electrolyte compositions that include one or more compounds of the present disclosure. The electrolyte compositions may comprise (a) a solvent composition including one or more of the compounds according to formula (I); and (b) at least one electrolyte salt. The electrolyte compositions of the present disclosure exhibit excellent oxidative stability, and when used in high voltage electrochemical cells (such as rechargeable lithium ion batteries) provide outstanding cycle life and calendar life. For example, when such electrolyte compositions are used in an electrochemical cell with a graphitized carbon electrode, the electrolytes provide stable cycling to a maximum charge voltage of at least 4.5V and up to 6.0V vs. Li/Li⁺.

Electrolyte salts that are suitable for use in preparing the electrolyte compositions of the present disclosure include those salts that comprise at least one cation and at least one weakly coordinating anion (the conjugate acid of the anion having an acidity greater than or equal to that of a hydrocarbon sulfonic acid (for example, PF₆⁻ anion or a bis(perfluoroalkanesulfonyl)imide anion); that are at least partially soluble in a selected compound of formula (I) (or in a blend thereof with one or more other compounds of formula (I) or one or more conventional electrolyte solvents); and that at least partially dissociate to form a conductive electrolyte composition. The salts may be stable over a range of operating voltages, are non-corrosive, and may be thermally and hydrolytically stable. Suitable cations include alkali metal, alkaline earth metal, Group JIB metal, Group IIIB metal, transition metal, rare earth metal, and ammonium (for example, tetraalkylammonium or trialkylammonium) cations, as well as a proton. In some embodiments, cations for battery use include alkali metal and alkaline earth metal cations. Suitable anions include fluorine-containing inorganic anions such as (FSO₂)₂N⁻, BF₄⁻, PF₆⁻, AsF₆⁻, and SbF₆⁻; CIO₄⁻; HSO₄⁻; H₂PO₄⁻; organic anions such as alkane, aryl, and alkaryl sulfonates; fluorine-containing and nonfluorinated tetraarylborates; carboranes and halogen-, alkyl-, or haloalkylsubstituted carborane anions including metallocarborane anions; and fluorine-containing organic anions such as perfluoroalkanesulfonates, cyanoperfluoroalkanesulfonylamides, bis(cyano)perfluoroalkanesulfonylmethides, (perfluoroalkanesulfonyl)imides, bis(perfluoroalkanesulfonyl)methides, and tris(perfluoroalkanesulfonyl)methides; and the like. Preferred anions for battery use include fluorine-containing inorganic anions (for example, (FSO₂)₂N⁻, BF₄⁻, PF₆⁻, and AsF₆⁻) and fluorine-containing organic anions (for example, perfluoroalkanesulfonates, bis(perfluoroalkanesulfonyl)imides, and tris(perfluoroalkanesulfonyl)methides). The fluorine-containing organic anions can be either fully fluorinated, that is perfluorinated, or partially fluorinated (within the organic portion thereof). In some embodiments, the fluorine-containing organic anion is at least about 80 percent fluorinated (that is, at least about 80 percent of the carbon-bonded substituents of the anion are fluorine atoms). In some embodiments, the anion is perfluorinated. The anions, including the perfluorinated anions, can contain one or more catenary heteroatoms such as, for example, nitrogen, oxygen, or sulfur. In some embodiments, fluorine-containing organic anions include perfluoroalkanesulfonates, bis(perfluoroalkanesulfonyl)imides, and tris(perfluoroalkanesulfonyl)methides.

In some embodiments, the electrolyte salts may include lithium salts. Suitable lithium salts include, for example, lithium hexafluorophosphate, lithium bis(trifluoromethanesulfonyl)imide, lithium bis(perfluoroethanesulfonyl)imide, lithium tetrafluoroborate, lithium perchlorate, lithium hexafluoroarsenate, lithium trifluoromethanesulfonate, lithium tris(trifluoromethanesulfonyl)methide, lithium bis(fluorosulfonyl)imide (Li—FSI), and mixtures of two or more thereof.

The electrolyte compositions of the present disclosure can be prepared by combining at least one electrolyte salt and a solvent composition including at least one compound of formula (I), such that the salt is at least partially dissolved in the solvent composition at the desired operating temperature. The compounds of the present disclosure (or a normally liquid composition including, consisting, or consisting essentially thereof) can be used in such preparation.

In some embodiments, the electrolyte salt is employed in the electrolyte composition at a concentration such that the conductivity of the electrolyte composition is at or near its maximum value (typically, for example, at a Li molar concentration of around 0.1-4.0 M, or 1.0-2.0 M, for electrolytes for lithium batteries), although a wide range of other concentrations may also be employed.

In some embodiments, one or more conventional electrolyte solvents are mixed with the compound(s) of formula (I) (for example, such that the compound(s) of formula (I) constitute from about 1 to about 80 or 90 percent of the resulting solvent composition). Useful conventional electrolyte solvents include, for example, organic and fluorine-containing electrolyte solvents (for example, propylene carbonate, ethylene carbonate, dimethyl carbonate, diethyl carbonate, ethyl methyl carbonate, dimethoxyethane, 7-butyrolactone, diglyme (that is, diethylene glycol dimethyl ether), tetraglyme (that is, tetraethylene glycol dimethyl ether), monofluoroethylene carbonate, vinylene carbonate, ethyl acetate, methyl butyrate, tetrahydrofuran, alkyl-substituted tetrahydrofuran, 1, 3-dioxolane, alkyl-substituted 1, 3-dioxolane, tetrahydropyran, alkyl-substituted tetrahydropyran, and the like, and mixtures thereof). Other conventional electrolyte additives (for example, a surfactant) can also be present, if desired.

The present disclosure further relates to electrochemical cells (e.g., fuel cells, batteries, capacitors, electrochromic windows) that include the above-described electrolyte compositions. Such an electrochemical cell may include a positive electrode, a negative electrode, a separator, and the above-described electrolyte composition.

A variety of negative and positive electrodes may be employed in the electrochemical cells. Representative negative electrodes include graphitic carbons e.g., those having a spacing between (002) crystallographic planes, d₀₀₂, of 3.45 Å>d₀₀₂>3.354 Å and existing in forms such as powders, flakes, fibers or spheres (e.g., mesocarbon microbeads); Li_{4/3}Ti_{5/3}0₄ the lithium alloy compositions described in U.S. Pat. No. 6,203,944 (Turner et al.) and U.S. Pat. No. 6,255,017 (Turner); and combinations thereof. Representative positive electrodes include LiFePO₄, LiMnPO₄, LiCoPO₄, LiMn₂O₄, LiCoO₂ and combinations thereof. The negative or positive electrode may contain additives such as will be familiar to those skilled in the art, e.g., carbon black for negative electrodes and carbon black, flake graphite and the like for positive electrodes.

The electrochemical devices of the present disclosure can be used in various electronic articles such as computers, power tools, automobiles, telecommunication devices, and the like.

Exemplary embodiments of the present disclosure include, but are not limited to, the following:

Embodiment 1. A fluorinated diaminoolefin of formula (I)

(R_{f}¹CF₂)(R_{f}²)NCH₂CH═CHCH₂N(R_{f}⁴)(CF₂R_{f}³)

where:
R_{f}¹ and R_{f}³, are independently selected from F, a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; and R_{f}² and R_{f}⁴ are independently selected from a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; or
at least one of (i) —CF₂R_{f}¹ and R_{f}² or (ii) —CF₂R_{f}³ and R_{f}⁴ are bonded together to form a fluorinated ring structure comprising 4-8 carbon atoms and optionally comprising at least one catenated atom selected from O, N, S or combinations thereof.

Embodiment 2. The fluorinated diaminoolefin of embodiment 1, wherein at least one of N(—CF₂R_{f}¹)(R_{f}²) or N(—CF₂R_{f}³)(R_{f}⁴) is a perfluorinated morpholino group.

Embodiment 3. The fluorinated diaminoolefin of embodiment 1, wherein at least one of N(—CF₂R_{f}1)(R_{f}2) or N(—CF₂R_{f}3)(R_{f}4) is a perfluorinated piperazino group.

Embodiment 4. The fluorinated diaminoolefin of embodiment 1, wherein at least one of N(—CF₂R_{f}¹)(R_{f}²) or N(—CF₂R_{f}³)(R_{f}⁴) is a perfluorinated pyrrolidyl group.

Embodiment 5. The fluorinated diaminoolefin of embodiment 1, wherein at least one of R_{f}¹, R_{f}², R_{f}³, or R_{f}⁴ is —CF₃, —CF₂CF₃, —CF₂CF₂CF₃ or —CF₂CF₂CF₂CF₃.

Embodiment 6. The fluorinated diaminoolefin of any one of the previous embodiments, wherein the fluorinated diaminoolefin is in a cis configuration.

Embodiment 7. The fluorinated diaminoolefin of any one of the previous embodiments, wherein the fluorinated diaminoolefin is in a trans configuration.

Embodiment 8. The fluorinated diaminoolefin of any one of the previous embodiments, wherein the fluorinated diaminoolefin comprises at least one of the following:

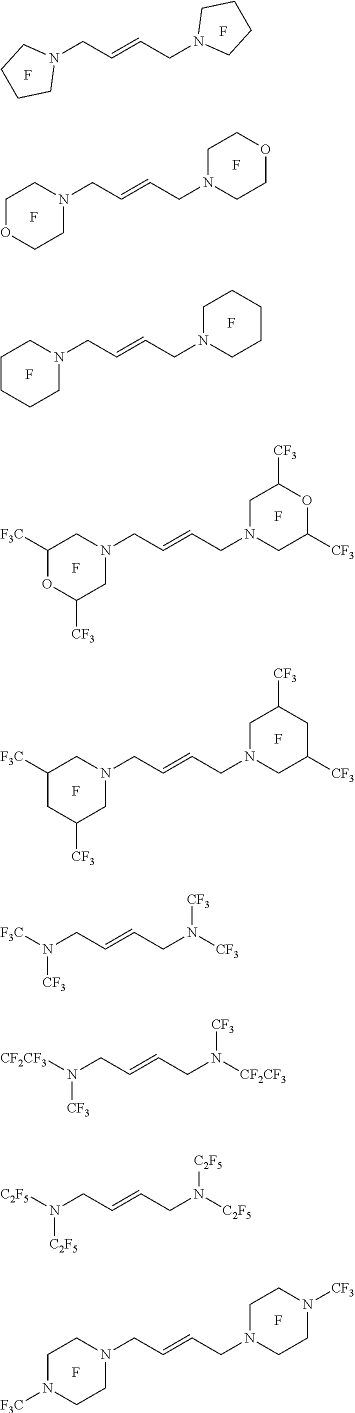

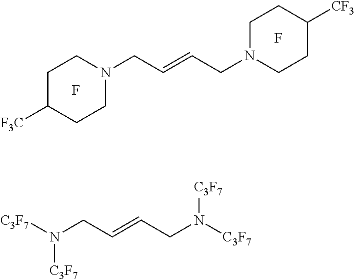

Embodiment 9. The fluorinated diaminoolefin of any one of the previous embodiments, wherein the fluorinated diaminoolefin is nonflammable based on closed-cup flashpoint testing following ASTM D-327-96 e-1.

Embodiment 10. The fluorinated diaminoolefin of any one of the previous embodiments, wherein the fluorinated diaminoolefin has a global warming potential of less than 100.

Embodiment 11. The fluorinated diaminoolefin of any one of the previous embodiments, wherein the fluorinated diaminoolefin has a boiling point of 170-250° C.

Embodiment 12. A composition comprising a purified form of the fluorinated diaminoolefin according to any one of embodiments 1-11.

Embodiment 13. A working fluid comprising the fluorinated diaminoolefin according to any one of the previous embodiments, wherein the fluorinated diaminoolefin is present in the working fluid in an amount of at least 25% by weight based on the total weight of the working fluid.

Embodiment 14. The working fluid of embodiment 13, wherein the working fluid further comprises a co-solvent.

Embodiment 15. Use of the fluorinated diaminoolefin of any one embodiments 1-11, wherein the fluorinated diaminoolefin is in a cleaning composition.

Embodiment 16. Use of the fluorinated diaminoolefin of any one embodiments 1-11, wherein the fluorinated diaminoolefin is an electrolyte solvent or additive.

Embodiment 17. Use of the fluorinated diaminoolefin of any one embodiments 1-11, wherein the fluorinated diaminoolefin is a heat transfer fluid.

Embodiment 18. Use of the fluorinated diaminoolefin of any one embodiments 1-11, wherein the fluorinated diaminoolefin is a vapor phase soldering fluid.

Embodiment 19. An apparatus for heat transfer comprising:

a device; and

a mechanism for transferring heat to or from the device, the mechanism comprising a heat transfer fluid that comprises the fluorinated diaminoolefin according to any one of embodiments 1-11.

Embodiment 20. An apparatus for heat transfer according to embodiment 19, wherein the device is selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, and a laser.

Embodiment 21. An apparatus according to any one of embodiments 19-20, wherein the mechanism for transferring heat is a component in a system for maintaining a temperature or temperature range of an electronic device.

Embodiment 22. An apparatus according to any one of embodiments 19-21, wherein the device comprises an electronic component to be soldered.

Embodiment 23. An apparatus according to any one of embodiments 19-22, wherein the mechanism comprises vapor phase soldering.

Embodiment 24. A method of transferring heat comprising:
providing a device; and
transferring heat to or from the device using a heat transfer fluid that comprises a fluorinated diaminoolefin according to any one of embodiments 1-11.

### EXAMPLES

All materials are commercially available, for example from Sigma-Aldrich Chemical Company, Milwaukee, Wis., USA, or known to those skilled in the art, unless otherwise stated or apparent.

The following abbreviations are used in this section: mL=milliliters, g=grams, h=hours, d=days, mol=mole, mmol=millimole, ° C.=degrees Celsius, GC=gas chromatography, GC-MS=gas chromatography-mass spectrometry, GC-FID=gas chromatography-flame ionization detection, Torr=torr, kPa=kilopascal. Abbreviations for materials used in this section, as well as descriptions of the materials, are provided in the Materials Table.

Materials Table

| Material | Description |
|---|---|
| (E)-1,4-dibromo-2-butene | Commercially available from Sigma-Aldrich Chemical |
| | Company |
| (Z)-1,4-dichloro-2-butene | Commercially available from Sigma-Aldrich Chemical |
| | Company |
| Aliquat 336 | Methyltrioctylammonium chloride, commercially available |
| | from Alfa Aesar, Ward Hill, MA, USA |
| KF | Potassium fluoride, spray dried, commercially available |
| | from Sigma-Aldrich Chemical Company |
| CsF | Cesium fluoride, commercially available from Alfa Aesar |
| KI | Potassium iodide, commercially available from Alfa Aesar |
| DMF | N,N-Dimethylformamide, commercially available from |
| | Sigma-Aldrich Chemical Company |
| NMP | N-Methylpyrrolidinone, commercially available from |
| | Sigma-Aldrich Chemical Company |
| Tetraglyme | Tetraethylene glycol dimethyl ether, commercially |
| | available from Alfa Aesar |
| DCM | Dichloromethane, commercially available from Sigma- |
| | Aldrich Chemical Company |
| Activated carbon | Commercially available from Sigma-Aldrich Chemical |
| | Company |
| 4 Angstrom molecular sieves | Commercially available from Sigma-Aldrich Chemical |
| | Company |
| K₂CO₃ | Potassium carbonate, commercially available from Alfa |
| | Aesar |
| Basic alumina | Commercially available from Alfa Aesar |
| Silica gel | Commercially available from Sigma-Aldrich Chemical |
| | Company |
| 2,2,3,3,5,6,6-heptafluoro-3,6- | Compounds can be prepared as described in H. V. Rasika |
| dihydro-2H-1,4-oxazine; | Dias et al. Dalton Trans. 2011, 40, 8569; V. A. Petrov, G. |
| 2,2,3,3,4,4,5-heptafluoro-3,4- | G. Belen'kii, L. S. German Bulletin of the Academy of |
| dihydro-2H-pyrrole; and | Sciences of the USSR Division of Chemical Science 1985 |
| 2,2,3,3,3-Pentafluoro-N- | 34, 1789; and V. A. Petrov et al. Izvestiya Akademii Nauk |
| (perfluoropropyl)propanimidoyl | SSSR, Seriya Khimicheskaya 1989, 1, 122. |
| fluoride | |

### Example 1 (EX-1) Preparation of (E)-1,4-bis(perfluoromorpholino)but-2-ene

To a 600 mL Parr reactor were charged NMP (73 g), KF (18.2 g, 313 mmol), Aliquat 336 (5.1 g, 13 mmol), and (E)-1,4-dibromo-2-butene (30.2 g, 141 mmol). The reactor was sealed and stirring commenced. 2,2,3,3,5,6,6-Heptafluoro-3,6-dihydro-2H-1,4-oxazine (65.6 g, 311 mmol) was slowly added with constant internal pressure observed due to formation of the 2,2,3,3,5,6,6-Heptafluoro-3,6-dihydro-2H-1,4-oxazine-derived aza anion by reaction with KF. After complete addition of 2,2,3,3,5,6,6-Heptafluoro-3,6-dihydro-2H-1,4-oxazine, the reaction mixture was slowly raised to 80° C. After stirring for 2 d, the reaction mixture was allowed to cool to room temperature and was then washed with water (300 mL). Analysis of the crude fluorous phase by GC-FID (gas chromatography-flame ionization detection) revealed complete consumption of the 1,4-dibromo-2-butene starting material. Purification of the fluorous phase by concentric tube distillation under reduced pressure (104° C./20 Torr (2.7 kPa)) afforded (E)-1,4-bis(perfluoromorpholino)but-2-ene (40.1 g, 55% yield). GC-MS (gas chromatography-mass spectrometry) analysis confirmed formation of the desired (E)-1,4-bis(perfluoromorpholino)but-2-ene.

### Example 2 (EX-2) Preparation of (E)-1,4-bis(perfluoromorpholino)but-2-ene

To a 600 mL Parr reactor were added 2,2,3,3,5,6,6-Heptafluoro-3,6-dihydro-2H-1,4-oxazine (46 g, 218 mmol), (E)-1,4-dibromo-2-butene (21 g, 98 mmol), DMF (207 g), KF (12.8 g, 220 mmol), KI (8.9 g, 53 mmol), and Aliquot 336 (1.0 g, 2.4 mmol). The reaction mixture was heated (75° C.) and stirred for 2 d. The resultant mixture was then allowed to cool to room temperature. GC analysis of the crude reaction material indicated formation of (E)-1,4-bis(perfluoromorpholino)but-2-ene (30.6 g, 61% yield). GC-MS analysis confirmed formation of the desired (E)-1,4-bis(perfluoromorpholino)but-2-ene.

### Example 3 (EX-3) Preparation of (E)-1,4-bis(perfluoropyrrolidin-1-yl)but-2-ene

To a 300 mL Parr reactor were added tetraglyme (52 g), (E)-1,4-dibromo-2-butene (25.2 g, 118 mmol), and CsF (38.4 g, 253 mmol). The reactor was sealed and then evacuated with stirring. To the resultant mixture was added 2,2,3,3,4,4,5-heptafluoro-3,4-dihydro-2H-pyrrole (49.4 g, 253 mmol). The reaction mixture was then slowly heated to 80° C. with stirring. After stirring for 16 h, the resultant mixture was allowed to cool to room temperature, diluted with water (100 mL), and then extracted with dichloromethane (2×100 mL). The resultant organic layer was concentrated under reduced pressure via rotavap. The crude residue was purified by single-plate distillation (65° C./3.5 torr (0.47 kPa)) to afford (E)-1,4-bis(perfluoropyrrolidin-1-yl)but-2-ene (49 g, 86% yield).

### Example 4 (EX-4) Preparation of (Z)-1,4-bis(perfluoromorpholino)but-2-ene

To a 600 mL stainless steel reaction vessel were added (Z)-1,4-dichloro-2-butene (20 g, 160 mmol), NMP (100 g), KF (30 g, 516 mmol), and KI (4 g, 24 mmol). The reaction vessel was then sealed and stirring commenced. 2,2,3,3,5,6,6-Heptafluoro-3,6-dihydro-2H-1,4-oxazine (70 g, 332 mmol) was then charged to the reaction and the resultant mixture was slowly heated to 85° C. followed by a 2 day stir. The resultant reaction mixture was then allowed to cool to room temperature followed by the addition of water (300 mL). The fluorous phase was then filtered and then analyzed by GC-FID indicating a mixture containing approximately 24% (Z)-1,4-bis(perfluoromorpholino)but-2-ene. Formation of (Z)-1,4-bis(perfluoromorpholino)but-2-ene was confirmed by GC-MS analysis.

### Application Example 1: Vapor Pressure of (E)-1,4-bis(perfluoromorpholino)but-2-ene

Vapor Pressure was measured using the stirred-flask ebuilliometer method described in ASTM E-1719-97 “Vapor Pressure Measurement by Ebuilliometry. The method used a 50-mL glass round-bottom flask. Vacuum was measured and controlled using a vacuum controller (J-KEM Scientific, Inc., St. Louis, Mo.). The pressure transducer was calibrated on the day of measurement by comparison with full vacuum and with an electronic barometer. The procedure began by slowly heating (E)-1,4-bis(perfluoromorpholino)but-2-ene, then applying vacuum until boiling occurred and a steady drop wise reflux rate was established. Pot temperature and pressure were recorded, then the vacuum controller was set for a higher absolute pressure and (E)-1,4-bis(perfluoromorpholino)but-2-ene was heated further until a new reflux point was established. The pressure level was raised in increments. The boiling point and vapor pressure data indicate that (E)-1,4-bis(perfluoromorpholino)but-2-ene would be useful in a heat transfer and vapor phase soldering application.

TABLE 2
Boiling Point and Vapor Pressure for
(E)-1,4-bis(perfluoromorpholino)but-2-ene

| Temp | Vapor Pressure of (E)-1,4-bis(perfluoromorpholino)but-2-ene | |
|---|---|---|
| (° C.) | (mmHg) | kPa |
| 54.8 | 0.9 | 0.1 |
| 83.8 | 5.3 | 0.71 |
| 109.8 | 20.2 | 2.69 |
| 148.4 | 99.0 | 13.2 |
| 181.5 | 299.4 | 39.9 |
| 215.1 | 739.1 | 98.5 |
| 215.8 | 760 | 101 |

### Application Example 2: Thermal Stability of (E)-1,4-bis(perfluoromorpholino)but-2-ene

The thermal stability was measured by charging a weighed amount of (E)-1,4-bis(perfluoromorpholino)but-2-ene (having a purity of 98.8%) into glass vials and then adding a weighed amount of absorbent. (The purity of the composition was measured by analyzing the sample using GC-FID and comparing the area of the peak of interest to the total peak areas in the chromatogram.) The mixture was stirred at 60° C. for 24 h and then analyzed by GC-FID. The percent purity of the treated sample was determined by the peak area of the (E)-1,4-bis(perfluoromorpholino)but-2-ene peak versus the total peak areas in the GC-FID chromatogram. The percent purity with the various absorbents are shown in Table 3 below. This data indicates that (E)-1,4-bis(perfluoromorpholino)but-2-ene would be useful for heat transfer and vapor phase soldering applications given its chemical and thermal stability in the presence of various absorbents.

TABLE 3
Thermal Stability of (E)-1,4-bis(perfluoromorpholino)but-2-ene

| | | | 4 Angstrom | | | |
|---|---|---|---|---|---|---|
| | No | Activated | Molecular | | Basic | Silica |
| | Adsorbent | Carbon | Sieves | K₂CO₃ | Alumina | Gel |
| GC | 98.8 | 98.8 | 98.8 | 98.9 | 99.5 | 98.8 |
| Purity | | | | | | |
| (%) | | | | | | |

Foreseeable modifications and alterations of this invention will be apparent to those skilled in the art without departing from the scope and spirit of this invention. This invention should not be restricted to the embodiments that are set forth in this application for illustrative purposes. To the extent that there is any conflict or discrepancy between this specification as written and the disclosure in any document mentioned or incorporated by reference herein, this specification as written will prevail.

1. A fluorinated diaminoolefin of formula (I)
(R_{f}¹CF₂)(R_{f}²)NCH₂CH═CHCH₂N(R_{f}⁴)(CF₂R_{f}³)
where:
R_{f}¹ is selected from a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; R_{f}³ is selected from F, a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; and R_{f}² and R_{f}⁴ are independently selected from a linear or branched perfluorinated alkyl group comprising 1-7 carbon atoms, or a linear or branched perfluorinated alkyl group comprising 1-7 carbon atoms comprising at least one catenated atom selected from O, N, S or combinations thereof; or
at least one of (i) —CF₂R_{f}¹ and R_{f}² or (ii) —CF₂R_{f}³ and R_{f}⁴ are bonded together to form a fluorinated ring structure comprising 4-8 carbon atoms and optionally comprising at least one catenated atom selected from O, N, S or combinations thereof.

2. The fluorinated diaminoolefin of claim 1, wherein at least one of N(—CF₂R_{f}¹)(R_{f}²) or N(—CF₂R_{f}³)(R_{f}⁴) is a perfluorinated morpholino group.

3. The fluorinated diaminoolefin of claim 1, wherein at least one of N(—CF₂R_{f}¹)(R_{f}) or N(—CF₂R_{f}³)(R_{f}⁴) is a perfluorinated piperazino group.

4. The fluorinated diaminoolefin of claim 1, wherein at least one of N(—CF₂R_{f}¹)(R_{f}) or N(—CF₂R_{f}³)(R_{f}⁴) is a perfluorinated pyrrolidyl group.

5. The fluorinated diaminoolefin of claim 1, wherein at least one of R_{f}¹, R_{f}², R_{f}³, or R_{f}⁴ is —CF₃, —CF₂CF₃, —CF₂CF₂CF₃ or —CF₂CF₂CF₂CF₃.

6. The fluorinated diaminoolefin of claim 1, wherein the fluorinated diaminoolefin is in a cis configuration.

7. The fluorinated diaminoolefin of claim 1, wherein the fluorinated diaminoolefin is in a trans configuration.

8. The fluorinated diaminoolefin of claim 1, wherein the fluorinated diaminoolefin comprises at least one of the following:

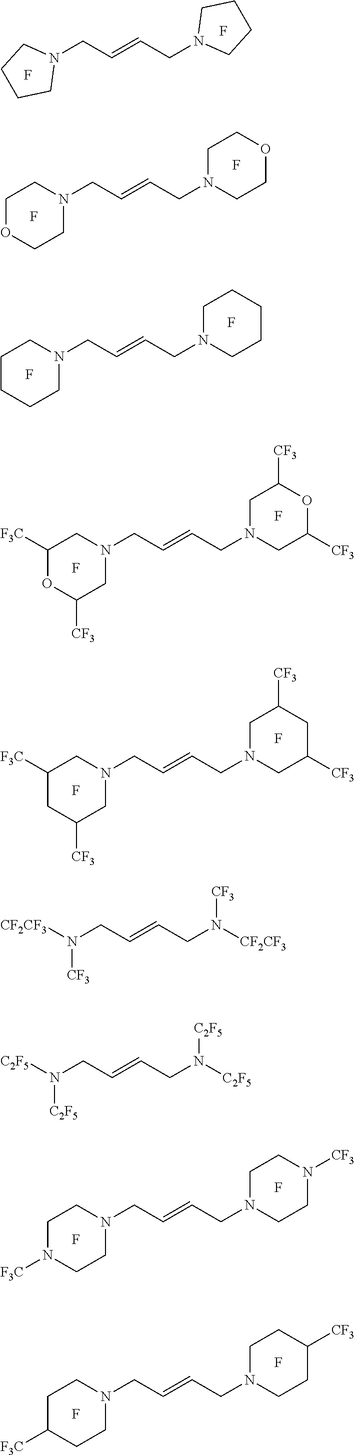

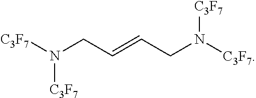

9. A composition comprising a purified form of the fluorinated diaminoolefin according to claim 1.

10. A working fluid comprising the fluorinated diaminoolefin according to claim 1, wherein the fluorinated diaminoolefin is present in the working fluid in an amount of at least 25% by weight based on the total weight of the working fluid.

11. Use of the fluorinated diaminoolefin of claim 1, wherein the fluorinated diaminoolefin is in a cleaning composition.

12. Use of the fluorinated diaminoolefin of claim 1, wherein the fluorinated diaminoolefin is an electrolyte solvent or additive.

13. Use of the fluorinated diaminoolefin of claim 1, wherein the fluorinated diaminoolefin is a heat transfer fluid.

14. Use of the fluorinated diaminoolefin of claim 1, wherein the fluorinated diaminoolefin is a vapor phase soldering fluid.

15. An apparatus for heat transfer comprising:
a device; and
a mechanism for transferring heat to or from the device, the mechanism comprising a heat transfer fluid that comprises the fluorinated diaminoolefin according to claim 1.

16. Use of the fluorinated diaminoolefin of claim 1, wherein the fluorinated diaminoolefin is nonflammable based on closed-cup flashpoint testing following ASTM D-327-96 e-1.

17. The fluorinated diaminoolefin of claim 1, wherein the fluorinated diaminoolefin has a boiling point of 170-250° C.

18. An apparatus for heat transfer according to claim 15, wherein the device is selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, and a laser.

19. A method of transferring heat comprising:
providing a device; and
transferring heat to or from the device using a heat transfer fluid that comprises a fluorinated diaminoolefin according to claim 1.
